(19) United States
(12) Patent Application Publication
(10) Pub. No.: US 2003/0035813 A1
(43) Pub. Date: Feb. 20, 2003

(54) IMMUNOLOGICAL TREATMENT METHODS AND AGENTS

(76) Inventors: ALBERT S. KLAINER, NEW YORK, NY (US); EMIL BISACCIA, BASKING RIDGE, NJ (US)

Correspondence Address: ROTHWELL, FIGG, ERNST & MANBECK, P.C., 1425 K STREET, N.W., SUITE 800, WASHINGTON, DC, 20005, US

(21) Appl. No.: 08/474,535

(22) Filed: Jun. 7, 1995

Related U.S. Application Data
(63) Continuation of application No. 08/089,430, filed on Jul. 9, 1993, now abandoned.
Application No. 08/089,430 is a continuation-in-part of application No. 08/086,553, filed on Jul. 1, 1993, now abandoned.
Application No. 08/086,553 is a continuation-in-part of application No. 07/809,590, filed on Dec. 17, 1991, now Pat. No. 5,284,869.
Application No. 07/809,590 is a continuation-in-part of application No. 07/993,908, filed on Dec. 18, 1992, now abandoned.
Application No. 07/993,908 is a continuation-in-part of application No. 07/460,811, filed on Jan. 4, 1990, now abandoned.
Application No. 07/460,811 is a continuation-in-part of application No. 07/295,454, filed on Jan. 10, 1989, now Pat. No. 4,960,408.
Application No. 07/295,454 is a continuation-in-part of application No. 07/364,063, filed on Jun. 8, 1989, now abandoned.

(51) Int. Cl. A61K 39/21; A01N 43/16; A61K 31/35

(52) U.S. Cl. 424/208.1; 514/455

(57) ABSTRACT

Methods, agents and compositions are provided for correcting immune response to a particular antigen or antigens. For example, the methods and agents disclosed herein can be used to stimulate an effective immune response against infection, e.g. an HIV infection. Alternatively, the inventive concepts embrace the inhibition of inappropriate immune response, one application of which is the prevention of restenosis following coronary angioplasty/arthrectomy.

### CROSS-REFERENCE

This application is a continuation-in-part of U.S. patent application Ser. No. 07/809,590, filed Dec. 17, 1991 and of Ser. No. 07/993,908, filed Dec. 18, 1992, which is a continuation of U.S. patent application Ser. No 07/460,811, filed Jan. 4, 1990 (abandoned), which is a continuation-in-part of U.S. patent application Ser. No. 07/295,454, filed Jan. 10, 1989 (now U.S. Pat. Nos. 4,960,408) and 07/364,063, filed Jun. 6, 1989 (abandoned), the disclosures of each of said applications and issued patent being incorporated herein by reference.

### FIELD OF THE INVENTION

The present invention relates to the field of immunology. More particularly, the invention relates to methods, agents and compositions for correcting immune response to a particular antigen or antigens. For example, the methods and agents disclosed herein can be used to stimulate an effective immune response against infection, e.g. an HIV infection. Alternatively, the inventive concepts embrace the inhibition of inappropriate immune response, one application of which is the prevention of restenosis following coronary angioplasty/arthrectomy.

### BRIEF DESCRIPTION OF THE INVENTION

An effective immune response generally includes a cellular response coupled with an antibody response. However, in order to have an effective immune response against a particular antigen, the antigen must be presented to the immune system in the proper context. So-called “up-regulation” of the immune system occurs when an antigen has been presented in a recognizable context thereby spurring the immune system into action. Conversely, “down-regulation” occurs as a result of the immune system's ability to self-govern its response to the particular antigen.

We have conducted extensive clinical work aimed at evaluating the potential for managing immune system response using a relatively new medical technique called photopheresis. Photopheresis is a technique wherein blood is treated extracorporally using a psoralen compound which is photoactivated by UVA irradiation. Its only current approved use is in the treatment of cutaneous T-cell lymphoma. However, we have evaluated and confirmed its potential in a number of areas, including AIDS therapy, Lymes disease and the prevention of restenosis following coronary angioplasty/arthrectomy.

Through our investigations we have discovered that photopheresis, when practiced in accordance with our disclosure, alters antigen presentation. In accordance with the invention antigenic peptides, polypeptides and/or native sub-units of infectious agents, for example, are obtained initially by subjecting a blood fraction from a donor, e.g. an AIDS patient, to photopheresis. The antigenic substances (peptides, polypeptides, native sub-units, etc.) may thereafter be reproduced through conventional replication/reproduction procedures that are known in the art.

The antigenic materials of the invention have utility both in up-regulating a deficient immune response as well as in down-regulating an inappropriate immune response.

### DETAILED DESCRIPTION OF THE INVENTION

While it is not intended that the scope of the present invention be limited by any specific theory of operation, it is believed that viral infections, particularly those which are not controlled by the normal immunological response of a patient, can be treated using a photopheresis treatment according to the invention. It is believed by the inventors that the photopheresis treatment according to the invention not only treats the viral infection, but is believed by the inventors to (i) restore the ability of a treated patient's immune system (which has been weakened by the viral infection) to combat other infections, including non-viral infections, and (ii) restore the immune system's anamnestic response to previous infections.

The photopheresis treatment method according to the invention is of particular value in the treatment of frequently mutating viral infections, such as retroviruses, for instance HIV retroviruses. In accordance with the photopheresis methods of the invention, treated infected cells as well as killed and/or attenuated virus, peptides, native sub-units of the virus itself (which are released upon cell break-up and/or shed into the blood) and/or pathogenic noninfectious viruses may be used.

Mutation of the viral antigen does not shield it from attenuation/inactivation during photopheresis and consequent generation of an immune response to the mutant forms of the viral antigen. Thus, the treatment methods according to the invention provide a dynamic autogenous vaccine against viral infections.

The inventive methods have been found by the inventors to be useful in the treatment of patients having a virus infection and who have an abnormally low white blood cell count and are particullarly useful in treating HIV retrovirus infections. The inventive methods are also particullarly useful for treating patients who are AIDS Carriers or who have AIDS or AIDS Related Complex.

According to the claimed methods, a photoactive compound is first administered to the blood of a patient who is infected with a virus. The photoactive compound may be administered in vivo (e.g. orally or intravenously) or may be administered in vitro to a portion of the patient's blood which has been removed from the patient by employing conventional blood withdrawal techniques.

Alternatively, free virus is isolated from infected cells using conventional virus isolation methods which are known in the art. The photoactive compound can be administered to the infected cells prior to virus isolation or can be administered to the free isolated virus. In the case of treating HIV infection, however, it is presently preferred to use both treated virus and treated virus infected cells in the methods described hereinbelow.

In accordance with the present invention, the photoactive compound selected should preferably be one that binds, in the case of a virus infected cell, to the cell membrane (e.g., by binding to a receptor and/or a nucleic acid fragment on the cell membrane) and/or to nucleic acid in the cell nucleus or cell cytoplasm, or, in the case of either free virus or cell associated virus, that binds to the virus surface (e.g., to a receptor and/or to a nucleic acid fragment on the virus surface) and/or to nucleic acid (e.g., DNA or RNA) which is incorporated in the virus, upon activation by exposure to electromagnetic radiation of a prescribed spectrum, such as ultraviolet light, for the purpose of inactivating and/or attenuating the virus and permitting the so treated virus and/or virus infected cells to be presented to the immune system of the patient. Psoralen compounds are particularly preferred for this purpose, especially the compound 8-methoxypsoralen—in which case UVA radiation is preferred for activating said compound.

Next, the portion of the patient's blood, or the free isolated virus, to which the photoactive compound has been administered is treated by subjecting the portion of the blood, or the free isolated virus, to photopheresis using said electromagnetic radiation—for example, ultraviolet light. The photopheresis step is preferably carried out in vitro using an extracorporeal photopheresis apparatus.

The photopheresis step in accordance with the present invention may also be carried out in vivo.

A presently preferred extracorporeal photopheresis apparatus for use in the methods according to the invention is currently manufactured by Therakos, Inc., Westchester, Pa. under the name UVAR. A description of the Therakos UVAR photopheresis apparatus may be found in U.S. Pat. No. 4,683,889, granted to R. L. Edelson on Aug. 14, 1987, the contents of which are hereby incorporated by reference in their entirety.

The exposure of blood, or free isolated virus, to ultraviolet light in a photopheresis apparatus is within the ability of persons having ordinary skill in the art.

When the photopheresis step is carried out in vitro, at least a fraction of the treated blood, or the treated free isolated virus, is returned to the patient following the photopheresis treatment. Preferably, the treatment method described hereinabove is repeated at an interval of about once per week to about once every four weeks. Most preferably, in the treatment of HIV infection, the treatment methods described herein are administered on two successive days and repeated approximately once per month (ie, the patient preferably receives two treatments every month).

In view of the disclosure contained herein, those persons who are skilled in the art will be able to adjust the treatment parameters—ie, dosage of the photoactive compound and electromagnetic radiation, periodicity of treatment (e.g., monthly, weekly, etc.) and the number of treatments administered in each period (e.g., twice per month on two successive days)—depending on the condition of the patient and the patient's response to the treatment.

Preferred photoactive compounds for use in accordance with the present invention are compounds known as psoralens (or furocoumarins) which are described in U.S. Pat. No. 4,321,919 the disclosure of which is incorporated herein by reference in their entirety.

The preferred photoactive compounds for use in accordance with the present invention include the following:

psoralen;

8-methoxypsoralen;

4,5′8-trimethylpsoralen;

5methoxypsoralen;

4-methylpsoralen;

4,4-dimethylpsoralen;

4-5′-dimethylpsoralen; and

4′, 8-methoxypsoralen

The most particularly preferred photoactive compound for use in accordance with the invention is 8-methoxypsoralen.

The determination of an effective dosage for in vitro virus inactivation of free isolated virus is within the ability of persons having ordinary skill in the art.

The photoactive compound, when administered to the patient's blood in vivo is preferably administered orally, but also can be administered intravenously and/or by other conventional administration routes.

The preferred dosage of the photoactive compound is in the range of about 0.3 to about 0.7 mg/kg of body weight although larger or smaller doses may be employed. When the photoactive compound is administered in vitro to only a portion of the patient's blood or fraction thereof, it is within the ability of those skilled in the art to calculate a dosage which is equivalent to said range based upon the volume of treated blood or fraction thereof.

In particular, when treating blood, blood components or some fraction thereof, (e.g. plasma, red cells, white cells, platelets, proteins or carrier proteins, etc.) that possibly contains a free or cell-associated virus such as HIV, it is especially preferred to treat same in vitro using a psoralen dosage within the range of 5 to 20 micrograms/ml, more preferably 5 to 10 micrograms/ml, most preferably about 10 micrograms/ml. Higher dosages may be employed if desired. The treatment does not require the use of a non-oxidizing atmosphere. It is preferred to carry out the treatment in the presence of oxidizing species that are either normally present in the blood, blood component or fraction thereof, or which are generated in situ (e.g. singlet oxygen, free radicals etc.). Additional oxidizing species may be employed if desired without hindering the treatment and which may even be additive. This treatment is capable of killing or inactivating or at least attenuating a substantial percentage of the loads of free or cell-associated virus present. It is preferred, especially in the case of HIV, to reduce the viral load at least 10%, more preferably at least 30%, most preferably at least about 90%. The treated blood may be administered to either an infected or a non-infected recipient. In either case, the treated blood may engender an immune response which is either protective against infection or additive in the case of an already infected person.

When administered orally, the photoactive compound should preferably be administered at least about one hour prior to the photopheresis treatment and no more than about three hours prior to the photopheresis treatment. The timing of administration may be adjusted up or down as needed depending on the bioavailability of the photoactive compound, its expected half-life, etc. If administered intravenously, the times would generally be shorter.

The photopheresis treatment in the treatment methods according to the invention is preferably carried out using long wavelength ultraviolet light (UVA) at a wavelength within the range of 320 to 400 nm. The exposure to ultraviolet light during the photopheresis treatment preferably has a duration of about three to four hours, although shorter or longer treatment periods may be used if desired.

Whatever the spectrum of electromagnetic radiation, the exposure of virus infected cells and/or virus thereto, following administration of the photoactive compound, should be of sufficient intensity/duration to effectively inactivate and/or attenuate the virus. The selection of an appropriate wavelength for photopheresis as well as the exposure, depending upon the photoactive compound being employed and the conditions of treatment (e.g., in vivo exposure or in vitro exposure), is within the ability of those skilled in the art in view of the present disclosure.

When the photoactive compound is 8-methoxypsoralen, it is preferred in accordance with the invention to utilize an exposure to UVA radiation of about 2 Joules/meter² based upon the surface area of the virus and virus infected cells undergoing treatment.

When the photopheresis treatment according to the invention is carried out in vivo, careful attention should be paid to controlling the maximum radiant exposure so as to avoid unnecessary injury to the patient. Methods for calculating maximum radiant exposure to ultraviolet light are known in the art and, therefore, shall not be described herein.

In summary, the invention provides a novel treatment for patients who are infected by a virus and who have depressed immune systems as a result of such infection, as well as for patients who are infected with an HIV retrovirus or who are AIDS Carriers or who have AIDS or AIDS Related Complex. Such patients cannot tolerate a treatment that would depress their immune systems.

The treatment methods according to the invention have been found by the inventors to be safe in this latter regard while also being effective in combatting HIV infection in humans.

The invention also provides methods for making vaccines. According to the invention, a donor who is infected with a virus, such as an HIV retrovirus, may be utilized to produce a vaccine against his infection as follows.

First, a photoactive compound as described hereinabove is administered to at least a portion of the donor's blood containing free virus and/or virus infected cells either prior to removal of the blood, either orally or intravenously, or after removal from the donor in which case it is administered in vitro. Optionally, a portion of the donor's blood could first be processed using known methods to substantially remove the erythrocytes and the photoactive compound is then administered to the resulting fraction.

In any case, the portion of blood (eg., an enriched leukocyte fraction thereof) and/or free isolated virus to which the photoactive compound has been administered is subjected to a photopheresis treatment using electromagnetic radiation of a prescribed spectrum, e.g.,ultraviolet light, preferably UVA, in the manner previously described. The treated blood, the treated portion thereof or the treated free isolated virus (as the case may be) is then administered back to the donor as an autogenous vaccine. It will be understood that in accordance with the present invention the treated virus can also be isolated from the treated blood or portion thereof following photopheresis treatment for use as a vaccine.

Additionally, in accordance with the present invention, the treated blood, which is itself a mixture of various blood components including peptides or polypeptides, eg., cytokines, lymphokines, monokines, etc., and/or the treated portion of blood may be processed, as is within the ability of persons having ordinary skill in the art, to isolate a particular component or components which may be used in the treatment of the virus infection of the donor and/or may be used as a vaccine against the virus.

Medicaments made using the photoactive compounds herein may be formulated using standard techniques which are already known in the art and, therefore, shall not be described in detail herein. By way of illustration, the photoactive compounds may be formulated using conventional excipients in the form of tablets, capsules and the like which would be suitable for oral administration. Alternatively the photoactive compounds described herein may, if desired, be formulated for parenteral administration by intravenous or intramuscullar routes. The medicaments can also be formulated as injectable solutions or suspensions for in vitro administration to a blood fraction which has been removed from an infected donor.

More particularly, the invention concerns antigenic peptides, polypeptides and/or native sub-units of infectious agents, for example, which are obtained initially by subjecting a blood fraction from a donor, e.g. an AIDS patient, to photopheresis. The antigenic substances (peptides, polypeptides, native sub-units, etc.) may thereafter be reproduced through conventional replication/reproduction procedures that are known in the art.

The antigenic materials of the invention have utility both in up-regulating a deficient immune response as well as in down-regulating an inappropriate immune response.

In the case of HIV, the antigenic material is a peptide, polypepetide or native sub-unit which is derived from a blood fraction taken from an HIV-infected host that has been treated by a photoactivated psoralen compound. The peptide, polypeptide or native sub-unit is characterized by a molecular weight within the range of about 17,000 to 160,000 daltons and an ability to stimulate an effective immune response to HIV infection.

More particularly, the peptide, polypeptide or native sub-unit is a protein or glycoprotein having a molecular weight of about 17 kd, 24 kd, 31 kd, 41 kd, 51 kd, 55 kd, 66 kd, 120 kd or 160 kd. Proteins or glycoproteins having a molecular weight of about 24 kd or 120 kd are particularly preferred. We found that seven HIV patients receiving the antigenic materials derived in accordance with our invention, developed a virus-specific cytotoxic T-cell response (>10% cytolysis) to p24 and gp120 antigen. More cytolysis was directed to p24 than to gp120.

HIV-infected individuals, prior to progression to AIDS, can mount an immune response through development of cytotoxic T cells (CTL), specific for HIV encoded peptides. To determine if ARC patients treated with photochemotherapy develop HIV-specific CTL responses, autologous Epstein-Barr virus (EBV) B cell lines were infected with recombinant vaccinia vectors that expressed the HIV gag or env proteins, and used as targets in a cytotoxicity assay.

Peripheral blood B cells from 7 ARC patients treated with photochemotherapy were immortalized by infection with EBV. The B cell lines were infected with vaccinia vectors: VV:gag (p24), vPE16 (gp120 and gp41), and vSC8 (vaccinia control). Cell surface expression was confirmed by Western blotting. Vaccinia infected B cells were labeled with ⁵¹chromium. Peripheral lymphocytes cryopreserved from various timepoints during the patients course were used as unstimulated cytotoxic effectors at effector to target ratios from 50:1 to 10:1.

Four of 7 patients developed virus-specific CTL (>10% cytolysis) during their treatment course. More cytolysis was directed to P24 (X=35%±22) than GP120 (X=22%±14) HIV antigen. Three of the 4 patients increased their CTL response during the 19-32 mo monitoring period, while 1 remained stable.

In the case of restenosis following coronary angioplasty, we believe that an aberrant immune response is implicated and that it is probably directed towards a new protein exposed through tissue damage. The antigenic substances of interest in this context, include cardiolipin, heat shock protein and endothelial antigen. It is noted that anticardiolipin antigen is observed in patients following angioplasty.

In this latter regard, a blood fraction which is obtained from a donor, who has previously undergone angioplasty/arthrectomy, is treated by photopheresis. The resulting antigenic material is capable of down-regulating subsequent immune response to thereby inhibit/prevent restenosis. In a controlled study, we found that the restenosis rate in patients receiving our described treatment, was only 14% which is significantly below the statiscal norm. One possible added benefit of photopheresis is its promotion of nitric oxide formation in the treated vessel which seems to be of possible benefit in further inhibiting restenosis.

It should be understood that while the foregoing description has been provided to illustrate the present inventions, it is not intended to limit the scope of the inventions as various modifications to the inventions described herein may be made by persons having ordinary skill in the art without departing from the spirit and scope thereof as defined in the following claims.

1. A peptide or native sub-unit which is derived from a blood fraction taken from an HIV-infected host that has been treated by a photoactivated psoralen compound, wherein the peptide or native sub-unit is characterized by a molecular weight within the range of about 17,000 to 160,000 daltons and an ability to stimulate an effective immune response to HIV infection.

2. The peptide or native sub-unit of claim 1, which is selected from the group consisting of proteins or glycoproteins having a molecular weight of about 17 kd, 24 kd, 31 kd, 41 kd, 51 kd, 55 kd, 66 kd, 120 kd and 160 kd.

3. The peptide or native sub-unit of claim 2, wherein the protein or glycoprotein has a molecular weight of about 24 kd.

4. The peptide or native sub-unit of claim 2, wherein the protein or glycoprotein has a molecular weight of about 120 kd.

5. An immunological composition comprising a peptide or native sub-unit which is derived from blood fraction taken from an HIV-infected host that has been treated by a photoactivated psoralen compound, wherein the peptide or native sub-unit is characterized by a molecular weight within the range of about 17,000 to 160,000 daltons and an ability to stimulate an effective immune response to HIV infection.

6. The immunological composition of claim 5, wherein the peptide or native sub-unit is selected from the group consisting of proteins or glycoproteins having a molecular weight of about 17 kd, 24 kd, 31 kd, 41 kd, 51 kd, 55 kd, 66 kd, 120 kd and 160 kd.

7. The immunological composition of claim 6, wherein the protein or glycoprotein has a molecular weight of about 24 kd.

8. The immunological composition of claim 6, wherein the protein or glycoprotein has a molecular weight of about 120 kd.

10. The immunological composition of claim 5, further comprising a supplemental adjuvant.

11. The immunological composition of claim 10, wherein the supplemental adjuvant is selected from the group consisting of Freunds adjuvant and incomplete Freunds adjuvant.
